(19) United States
(12) Patent Application Publication
(10) Pub. No.: US 2014/0026254 A1
(43) Pub. Date: Jan. 23, 2014

(54) Gene Implicated in Drought Stress Tolerance and Transformed Plants with the Same

(71) Applicant: INDUSTRY FOUNDATION OF CHONNAM NATIONAL UNIVERSIT, Gwangju (KR)

(72) Inventors: Cheol Soo KIM, Gwangju (KR); Hyun-Woo JU, Gwangju (KR); Ji-Hee MIN, Gwangju (KR)

(73) Assignee: INDUSTRY FOUNDATION OF CHONNAM NATIONAL UNIVERSITY, Gwangju (KR)

(21) Appl. No.: 13/687,087

(22) Filed: Nov. 28, 2012

(30) Foreign Application Priority Data
Jul. 17, 2012 (KR) 10-2012-0077892

(51) Int. Cl. C12N 15/82

(52) U.S. Cl. CPC C12N 15/8273
USPC 800/278; 435/419; 800/298

(57) ABSTRACT

The present invention relates to a composition for improving drought stress tolerance in a plant, a transgenic plant with enhanced drought stress tolerance, and a method for preparing a transgenic plant. The novel functional plant having excellent drought stress-tolerance may be prepared using the composition for improving drought stress tolerance and a method for preparing a transgenic plant.

### CROSS-REFERENCE TO RELATED APPLICATIONS

This application claims priority from Korean Patent Application No. 2012-0077892, filed on Jul. 17, 2012, in the Korean Intellectual Property Office, the disclosure of which is incorporated herein by reference.

### BACKGROUND

1. Field of the Invention

The present invention relates to a gene associated with drought stress tolerance and a transgenic plant in which the expression of the gene is suppressed.

2. Description of the Related Art

Protein ubiquitination is an important post-translational modification process that is employed by eukaryotes to regulate diverse cellular and developmental processes (Dye and Schulman, 2007). In higher plants, ubiquitinated proteins are involved in abiotic or biotic stress responses, hormone responses, cell cycle progression, and cell differentiation (Craig et al., 2009; Santner and Estelle, 2009; Marrocco et al., 2010; Ryu et al., 2010). Ubiquitin (Ub), a highly conserved 8-kDa protein, is first activated by the Ub-activating enzyme E1 in an ATP-dependent manner and is transferred to the Ub-conjugating enzyme E2. The Ub-E2 complex then binds Ub-protein ligase E3 that promotes the transfer of Ub from the Ub-E2 to a substrate protein, which is then recognized and degraded by the 26S proteasome (Vierstra, 2003). Apart from directing proteolysis, there is also growing evidence for non-degradative functions of protein ubiquitination, such as DNA repair and protein trafficking (Chen et al., 2009). In the Arabidopsis genome, more than 1,400 genes are predicted to encode different potential Ub-E3 ligases (Vierstra, 2009). E3 ligases can be classified into two groups. One class consists of RING (for Really Interesting New Gene)/U-BOX and HECT (for Homology to E6-AP Carboxyl Terminus) E3 enzymes that act as a single subunit. The other class that includes SCF (for Skp1-Cullin-F box) and APC (for Anaphase-Promoting Complex) functions as a multi-subunit complex (Gmachl et al., 2000; Tyers and Jorgensen, 2000; Lin et al., 2002). There are about 469 RING motif-containing E3 ligases, which comprise the third largest gene family in Arabidopsis (Mudgil et al., 2004; Stone et al., 2005). The Cys-rich RING finger was first described in the early 1990s (Freemont et al., 1991). It is defined as a linear series of conserved Cys and His residues (C3HC/HC3) that bind two zinc atoms in a cross brace arrangement. RING fingers can be divided into two types, C3HC4 (RING-HC) and C3H2C3 (RING-H2), depending on the presence of either a Cys or a His residue in the fifth position of the motif (Freemont, 2000). Recently, a number of Arabidopsis RING E3 ligases were shown to be involved in various cellular processes, such as auxin signaling, abscisic acid signaling, brassinosteroid response, seed germination, seedling development, adaptive pathway to nitrogen limitation, and sugar responses (Stone et al., 2006; Peng et al., 2007; Bu et al., 2009; Santner and Estelle, 2009; Huang et al., 2010). In particular, RING proteins play a key role in the response to environmental stimuli. For example, they participate in photomorphogenesis, defense signaling, senescence, tolerance mechanisms against cold, drought, salt, and osmotic stress (Yan et al., 2003; Craig et al., 2009; Fujita et al., 2011; Smirnova et al., 2011).

Throughout this application, various publications and patents are referred and citations are provided in parentheses. The disclosures of these publications and patents in their entities are hereby incorporated by references into this application in order to fully describe this invention and the state of the art to which this invention pertains.

### SUMMARY

The present inventors have made intensive studies to identify a gene for improving drought stress tolerance in a plant. As results, we have discovered that the activity of a protein consisting of the amino acid sequence of SEQ ID NO:2 or the expression of a nucleotide sequence of SEQ ID NO:1 encoding the amino acid sequence of SEQ ID NO:2 is involved in plant phenotype, and have demonstrated that the transgenic plant shows a significantly enhanced tolerance to drought stress where the activity of a protein consisting of the amino acid sequence of SEQ ID NO:2 is inhibited or the expression of a nucleotide sequence of SEQ ID NO:1 is suppressed in the plant.

Accordingly, it is an aspect of this invention to provide a composition for improving drought stress tolerance in a plant.

It is another aspect of this invention to provide a plant cell or a plant having drought stress tolerance.

It is still another aspect of this invention to provide a method for preparing a transgenic plant with enhanced drought stress tolerance.

It is still another aspect of this invention to provide a method for conferring drought stress tolerance on a plant.

Other aspects and advantages of the present invention will become apparent from the following detailed description together with the appended claims and drawings.

### BRIEF DESCRIPTION OF THE DRAWINGS

FIGS. 1A and 1B represent structural features of the AtRZF1 protein. FIG. 1A represents the structure of the conserved regions of the AtRZF1 protein. The prediction of a signal peptide is inconsistent between different software programs and is thus depicted with a question mark. The primary structure harbors RING-H2 zinc finger motif site (186-226) is shown in the black box. FIG. 1B represents alignment of C3H2C3-type RING motif deduced amino acid sequences of AtRZF1 and other AtRZF1 homologs from different plant species. Shown are the sequences of AtRZF1 (At3g56580), AtRHC1a (At2g40830), AtRHC2a (At2g39720), AtSIS3 (At3g47990), Oryza sativa RHC1a (OsRZF1; NP_{—}001044543) and Zea mays RHC1a (ZmRHC1a; NP_{—}001149547). Black and gray shading indicate identical and similar amino acids, respectively.

FIG. 2 shows alignment between deduced amino acid sequence of AtRZF1 and those of other AtRZF1 homologs. Shown are the sequences of AtRZF1 (At3g56580), OsRZF1 (NP_{—}001044543) and ZmRHC1a (NP_{—}001149547). Black and gray shading indicate identical and similar amino acids, respectively. Gaps are inserted for sequence alignment optimization. RING H2-type zinc finger domain (186-226) is indicated as a black line.

FIG. 3 shows subcellular localization of AtRZF1-EGFP fusion proteins. The 35S-EGFP and 35S-AtRZF1-EGFP constructs were transformed into Arabidopsis leaf protoplasts by a PEG-mediated method. Localization of fusion proteins were visualized by fluorescence microscopy. Scale bar denotes 10 μm.

FIG. 4 represents expression patterns of an AtRZF1 promoter-GUS construct in a transgenic Arabidopsis plant: (a) a 7-day-old seedling; (b) a full-expanded rosette leaf in a 3-week-old transgenic plant; (c) a flower in a 3-week-old transgenic plant; (d) anthers of stamen; and (e) pollens. Strong GUS activity was detected in pollens.

FIGS. 5A and 5B are graphs showing expression of the AtRZF1 gene in Arabidopsis under water deficit stress. The expression of AtRZF1 involved in mannitol (FIG. 5A) or drought (FIG. 5B) response is determined by quantitative real-time PCR analysis. Total RNA samples obtained from 14-day-old plants treated with drought or 400 mM mannitol at the indicated times. Error bars indicate standard deviations of three independent biological samples. Differences between the expression of AtRZF1 or RAB18 in 14-day-old Arabidopsis seedlings untreated and treated with various abiotic stresses are significant at the P<0.01 (**) levels. The RAB18 gene was used as a control for the drought or mannitol stress treatment.

FIGS. 6A and 6B are the results of assays for E3 ubiquitin ligase activity of AtRZF1 in vitro. Purified MBP-AtRZF1 was incubated at 37° C. for 1 h with E1, E2, ubiquitin (Ub), and ATP. Polyubiquitin chains were visualized with anti-ubiquitin (a) and anti-MBP (b) antibodies. Omission of E1 or E2 resulted in a loss of ubiquitination. MBP served as a negative control. Numbers on the left indicate the molecular masses of marker proteins in kDa.

FIG. 7 shows influence of atrzf1 mutant line on osmotic stress tolerance. (a) Expression levels of AtRZF1 in wild-type (WT), atrzf1 mutant, and two independent transgenic lines overexpressing AtRZF1 (OX1-1 and OX4-2) were determined by RT-PCR using total RNA isolated from 2-week-old seedlings. Actin8 was used as an internal control in RT-PCR. (b) Osmotic stress effect on cotyledon greening. Seeds were sown on MS agar plates supplemented without (−) or with (+) 400 mM mannitol and permitted to grow for 8 days, followed by counting seedlings with green cotyledons (triplicates, n=50 each). Error bars represent standard deviations. Differences among WT, mutant, and transgenic plants grown in the same conditions are significant at the P<0.01 (**) level.

FIG. 8 represents osmotic stress tolerance of atrzf1 mutant lines. Seeds were sown on MS agar plates supplemented without (−) or with (+) 400 mM mannitol and permitted to grow for 8 days. atrzf1 mutant lines show marked dark green leaves under osmotic condition compared with WT and AtRZF1-overexpressing transgenic lines (OX1-1 and OX4-2). Scar bar indicates 10 mm.

FIGS. 9A and 9B represent drought stress tolerance of atrzf1 mutant lines. atrzf1 mutant lines show distinguished dark green leaves and grow well under drought condition compared with WT and AtRZF1-overexpressing transgenic lines (OX1-1 and OX4-2). As for FIG. 9A, 2-week-old plants were grown for 10 days without watering (drought). As for FIG. 9B, Survival rate in plants was assessed 3 days after re-watering.

FIGS. 10A and 10B are results measuring water loss and electrolyte leakage in WT, atrzf1, and AtRZF1-overexpressing plants. FIG. 10A shows quantification of water loss in 2-week-old WT, atrzf1, and two independent AtRZF1-overexpressing plants. Rosette leaves of the same developmental stages were excised and weighed at various time points after detachment. Water loss was calculated as the percentage of initial fresh weight. Data represent average values ±SD of five leaves from each of seven replicates. The asterisk denotes a statistically significant difference compared with the wild-type [0.05>P>0.01 (*) or the P<0.01 (**)]. FIG. 10B shows electrolyte leakage of leaf cells of WT, atrzf1, and two independent AtRZF1-overexpressing transgenic plants under drought stress. Light-grown 2-week-old plants were grown for 10 days with (normal) or without (drought) watering. Leaf tissues were carefully excised after drought treatment, and used for measuring electrolyte leakage. Data represent average values ±SD of seven leaves from each of seven replicates. Differences among WT, mutant, and transgenic plants grown in the same conditions are significant at the 0.05>P>0.01 (*) or the P<0.01 (**) levels.

FIGS. 11A-11C show expression of stress-regulated genes (P5CS1, P5CR, RAB18, RD29A, RD29B, AOX1a, COR15A, ERD15, and ERD1). Light-grown 2-week-old WT, atrzf1, and two independent AtRZF1-overexpressing plants were further grown for 10 days with (normal) or withholding (drought) water. Total RNA was obtained from treated plants and analyzed by qPCR using gene-specific primers listed in Table 1. Each bar indicates the induction fold of the P5CS1, P5CR, RAB18, RD29A, RD29B, AOX1a, COR15A, ERD15, and ERD1 genes in response to drought stress as compared to the control treatment (normal condition). The mean value of three technical replicates was normalized to the levels of Actin8 mRNA, an internal control. Differences between the expression of P5CS1, P5CR, RAB18, RD29A, RD29B, AOX1a, COR15A, ERD15, and ERD1 in Arabidopsis seedlings untreated and treated with drought stress are significant at the 0.05>P>0.01 (*) or the P<0.01 (**) levels.

FIG. 12 is a result measuring leaf proline content in WT, atrzf1, and AtRZF1-overexpressing plants. Light-grown 2-week-old plants were grown for 10 days with (−) or without (+) watering. Leaf tissues were carefully excised after drought treatment, and used for measuring proline content. Error bars represent standard deviations. Differences among WT, mutant, and transgenic plants grown in the same conditions are significant at the P<0.01 (**) levels.

### DETAILED DESCRIPTION OF THIS INVENTION

In one aspect of this invention, there is provided a composition for improving drought stress tolerance in a plant, comprising an inhibitor against a protein consisting of the amino acid sequence of SEQ ID NO:2 or an expression inhibitor for the nucleotide sequence of SEQ ID NO:1 encoding the amino acid sequence of SEQ ID NO:2.

The present inventors have made intensive studies to identify a gene for improving drought stress tolerance in a plant. As results, we have discovered that the activity of a protein consisting of the amino acid sequence of SEQ ID NO:2 or the expression of a nucleotide sequence of SEQ ID NO:1 encoding the amino acid sequence of SEQ ID NO:2 is involved in plant phenotype, and have demonstrated that the transgenic plant shows a significantly enhanced tolerance to drought stress where the activity of a protein consisting of the amino acid sequence of SEQ ID NO:2 is inhibited or the expression of a nucleotide sequence of SEQ ID NO:1 is suppressed in the plant.

According to this invention, it would be obvious to the skilled artisan that a nucleotide sequence encoding the amino acid sequence of SEQ ID NO:2 is not limited to that of SEQ ID NO:1 listed in the appended Sequence Listings.

For nucleotides, the variations may be purely genetic, i.e., ones that do not result in changes in the protein product. This includes nucleic acids that contain functionally equivalent codons, or codons that encode the same amino acid (for example, six codons for arginine or serine corresponding on codon degeneracy), or codons that encode biologically equivalent amino acids.

Considering biologically equivalent variations described hereinabove, the nucleic acid molecule of this invention may encompass sequences having substantial identity to the sequence described in Sequence listings. Where the present sequence is aligned with arbitrary sequences in a maximal manner and the aligned sequences are analyzed using conventional alignment algorithms, the sequences having the substantial identity show at least 60%, more preferably at least 70%, much more preferably at least 80%, and most preferably at least 90% similarity to the nucleic acid molecule of this invention. Methods of alignment of sequences for comparison are well-known in the art. Various programs and alignment algorithms are described in: Smith and Waterman, Adv. Appl. Math. 2: 482 (1981); Needleman and Wunsch, J. Mol. Bio. 48: 443 (1970); Pearson and Lipman, Methods in Mol. Biol. 24: 307-31 (1988); Higgins and Sharp, Gene 73: 237-44 (1988); Higgins and Sharp, CABIOS 5: 151-3 (1989) Corpet et al., Nuc. Acids Res. 16: 10881-90 (1988); Huang et al., Comp. Appl. BioSci. 8: 155-65 (1992); and Pearson et al., Meth. Mol. Biol. 24: 307-31 (1994). The NCBI Basic Local Alignment Search Tool (BLAST) [Altschul et al., J. Mol. Biol. 215: 403-10 (1990)] is available from several sources, including the National Center for Biological Information (NBCl, Bethesda, Md.) and on the Internet, for use in connection with the sequence analysis programs blastp, blasm, blastx, tblastn and tblastx. BLAST can be accessed at http://www.ncbi.nlm.nih.gov/BLAST/. A description of how to determine sequence identity using this program is available at http://www.ncbi.nlm.nih.gov/BLAST/blast_help.html.

The term “drought stress” used herein refers to a condition without normal watering in plant growth, which is utilized as a very common term including all kind of abiotic (for example, treatment with diverse chemicals or exposure under dehydration conditions) or biotic (for example, infection caused by various sources) stresses that induce harmful effects on plant growth and survival.

The term to have a stress “tolerance” or “resistance” as used herein means to exhibit a strongly detectable tolerance against the aforementioned stress compared with damages in WT (wild-type), and the transgenic plant of this invention refers to a plant or part thereof, a plant tissue and a plant cell with the foregoing tolerance or resistance.

According to the present invention, the inhibitor against the protein of this invention means an inhibitor to suppress the activity of a E3 ubiquitin ligase AtRZF1 protein having the amino acid sequence of SEQ ID NO:2 or to interfere with the binding of AtRZF1 protein to a substrate thereof.

According to an embodiment, the inhibitor against the protein consisting of the amino acid sequence of SEQ ID NO:2 includes an antibody, a peptide aptamer, an AdNectin, an affibody, an Avimer, a Kunitz domain or a chemical compound.

The antibody inhibiting the activity of the AtRZF1 protein by specific binding to the AtRZF1 protein capable of being utilized in this invention may be polyclonal or monoclonal, preferably monoclonal. The antibody could be prepared according to conventional techniques such as a fusion method [Kohler and Milstein, European Journal of Immunology, 6: 511-519 (1976)], a recombinant DNA method (U.S. Pat. No. 4,816,56) or a phage antibody library [Clackson, et al., Nature, 352: 624-628 (1991) and Marks et al, J. Mol. Biol., 222:58, 1-597 (1991)]. The general procedures for antibody production are described in Harlow, E. and Lane, D., Using Antibodies: A Laboratory Manual, Cold Spring Harbor Press, New York, 1988; Zola, H., Monoclonal Antibodies: A Manual of Techniques, CRC Press, Inc., Boca Raton, Fla., 1984; and Coligan, CURRENT PROTOCOLS IN IMMUNOLOGY, Wiley/Greene, NY, 1991, which are incorporated herein by references. For example, the preparation of hybridoma cell lines for monoclonal antibody production is done by fusion of an immortal cell line and the antibody-producing lymphocytes. This can be carried out in a feasible manner by techniques well known in the art. Polyclonal antibodies may be prepared by injection of the AtRZF1 protein antigen to a suitable animal, and by collection of antiserum containing antibodies from the animal, followed by isolating specific antibodies therefrom by any of the known affinity techniques.

The peptide aptamer capable of being utilized in the present invention includes a DNA or RNA oligonucleotide having a special conformational folding to bind to a target antigen with high specificity and affinity. The peptide aptamer may be prepared by SELEX [Systemic Evolution of Ligands by Exponential Enrichment; Tuerk and Gold, Science, 249: 505-510 (1990)].

The chemical compound to be an inhibitor against a protein (AtRZF1 protein) consisting of the amino acid sequence of SEQ ID NO:2 in this invention may be obtained by screening a substance that suppresses the activity of AtRZF1 protein or binds to AtRZF1 protein. In this connection, AtRZF1 protein may be any form of AtRZF1 protein including a purified or cellular form.

The method to screen a chemical compound as a AtRZF1 protein inhibitor in the present invention may be carried out in a high-throughput manner according to various techniques such as diverse binding assays known to those ordinarily skilled in the art. In screening method, test substance or AtRZF1 protein may be labeled with a detectable label. For example, the detectable label includes chemical (e.g., biotin), enzyme (horseradish peroxidase, alkaline phosphatase, peroxidase, luciferase, β-galactosidase and β-glucosidase), radio isotope (e.g., C¹⁴, I¹²⁵, P³² and S³⁵), fluorescent [coumarin, fluorescein, FITC (fluorescein Isothiocyanate), rhodamine 6G, rhodamine B, TAMRA (6-carboxy-tetramethyl-rhodamine), Cy-3, Cy-5, Texas Red, Alexa Fluor, DAPI (4,6-diamidino-2-phenylindole), HEX, TET, Dabsyl and FAM], luminescent, chemiluminescent, FRET (fluorescence resonance energy transfer) or metal (for example, gold and silver) substances.

Using detectable labels linking to AtRZF1 protein or test substance, the binding of AtRZF1 protein to test substance may be determined by measuring a signal depending on labels. For example, in an alkaline phosphatase as a label of this invention the signal may be detected using bromochloroindolylphosphate (BCIP), nitro blue tetrazolium (NBT), naphthol-AS-B1-phosphate or ECF (enhanced chemifluorescence) substrate. Where a horseradish peroxidase is used as a label, the signal may be determined using chloronaphtol, aminoethylcarbazol, diaminobenzidine, D-luciferin, lucigenin (bis-N-methylacridinium nitrate), resorufin benzyl ether, luminol, Amplex Red reagent (10-acetyl-3,7-dihydroxyphenoxazine), HYR (p-phenylenediamine-HCl and pyrocatechol), TMB (tetramethylbenzidine), ABTS (2,2′-Azine-di[3-ethylbenzthiazoline sulfonate]), o-phenylenediamine (OPD) and naphtapyronine substrate.

Alternatively, the binding of test substance to AtRZF1 protein may be analyzed without labeling of interactants. For example, it may be analyzed using a microphysiometer whether test substance is bound to AtRZF1 protein. Microphysiometer based on LAPS (light-addressable potentiometric sensor) is an analytic instrument to measure a rate to acidify cell-surrounding environment. The change of acidification rate may be utilized as an indicator for the binding between test substance and AtRZF1 protein [McConnell, et al., Science 257: 1906-1912 (1992)].

The binding capacity of test substance with AtRZF1 protein may be determined by a real-time biomolecule interaction analysis (BIA) (Sjolander & Urbaniczky, Anal. Chem. 63:2338-2345 (1991), and Szabo, et al., Curr. Opin. Struct. Biol. 5: 699-705 (1995)). BIA is an analytic technique to quantify specific interactions without labeling of interactants in a real-time manner (for example, BIAcore™). The changes in surface plasmon resonance may be utilized as an indicator of real-time responses between molecules.

Meanwhile, the screening method may be carried out according to two-hybrid analysis or three-hybrid analysis (U.S. Pat. No. 5,283,317; Zervos, et al., Cell 72, 223-232, 1993; Madura, et al., J. Biol. Chem. 268, 12046-12054, 1993; Bartel, et al., BioTechniques 14, 920-924, 1993; Iwabuchi, et al., Oncogene 8, 1693-1696, 1993; and WO 94/10300). In this connection, AtRZF1 protein may be used as a bait protein. According to this method, it is possible to screen a substance bound to AtRZF 1 protein, particularly a protein. Two-hybrid system is based on a module property of a transcription factor consisting of divisible DNA-binding domain and activation domain. Briefly, this method utilizes two DNA constructs. For example, in one construct, AtRZF1-encoding polynucleotide is fused with a DNA-binding domain-encoding polynucleotide of well-known transcription factor (e.g., GAL-4). In the other construct, a DNA sequence encoding a protein of interest (“pray” or “sample”) is fused with a polynucleotide encoding an activation domain of the foregoing transcription factor. Where the complex is formed by in vivo interaction between the bait and the pray, DNA-binding domain and activation domain of transcription factor is adjacent, contributing to facilitating the transcription of a reporter gene (for example, LacZ). As a result, the expression of the reporter gene may be detectable, suggesting that the protein of interest may be bound to AtRZF1 protein, and thus utilized as an AtRZF1 protein inhibitor.

According to this invention, the expression inhibitor for the nucleotide sequence of SEQ ID NO:1 encoding the amino acid sequence of SEQ ID NO:2 preferably includes a nucleic acid molecule or a mutation-inducing agent.

The term “suppression of target gene expression” used herein in the specification refers to a modification on a nucleotide sequence causing functional reduction of a target gene, thereby leading to undetectable or meaningless expression level of the target gene, preferably.

According to an embodiment, the nucleic acid molecule of this invention includes siRNA, shRNA, miRNA, ribozyme, PNA (peptide nucleic acids) or antisense oligonucleotide.

The term “siRNA” used herein refers to a short RNA duplex that enables to mediate RNA interference via cleavage of target mRNA. The siRNA may consist of a sense RNA strand (having a sequence corresponding to a target mRNA sequence) and an antisense RNA strand (having a sequence complementary to a target mRNA sequence). The siRNA to inhibit expression of a target gene provides effective gene knock-down method or gene therapy method.

The siRNA of this invention is not restricted to a RNA duplex of which two strands are completely paired, and may comprise non-paired portion such as mismatched portion with non-complementary bases and bulge with no opposite bases. The overall length of the siRNA is 10-100 nucleotides, preferably, 15-80 nucleotides, and most preferably, 20-70 nucleotides. The siRNA may comprise either blunt or cohesive end so long as it enables to silent a target gene expression due to RNAi effect. The cohesive end may be prepared in 3′-end overhanging structure or 5′-end overhanging structure. The number of any end overhanging base may be not limited. For example, the number of base may include 1-8 bases, and preferably, 2-6 bases. In addition, siRNA may include a low molecular weight RNA (for example, natural RNA molecule such as tRNA, rRNA and viral RNA, or artificial RNA molecule) in the protruded portion of one end to the extent that it enables to maintain an effect in the inhibition of a target gene expression. The terminal structure of siRNA is not demanded as cut structure at both ends, and one end portion of double strand RNA may be stem-and-loop structure linked by a linker RNA. The length of linker is not restricted where it has no influence on the pair formation of the stem portion.

The term “shRNA” used herein means a single-strand nucleotide consisting of 50-70 bases, and forms a stem-loop structure in vovo. Long RNA of 19-29 nucleotides is complementarily base-paired at both directions of a loop consisting of 5-10 nucleotides, forming a double-stranded stem.

The term “miRNA (microRNA)” functions to regulate gene expression and means a single strand RNA molecule composed of 20-50 nucleotides in full-length, preferably 20-45 nucleotides, more preferably 20-40 nucleotides, much more preferably 20-30 nucleotides and most preferably, 21-23 nucleotides. The miRNA is an oligonucleotide which is not expressed intracellularly, and forms a short stem-loop structure. The miRNA has a whole or partial complementarity to one or two or more mRNAs (messenger RNAs), and the target gene expression is suppressed by the complementary binding of miRNA to the mRNA thereof.

The term used herein “ribozyme” refers to a RNA molecule having an activity of an enzyme in itself which recognizes and restricts a base sequence of a specific RNA. The ribozyme consists of a binding portion capable of specifically binding a base sequence complementary to a transfer RNA strand and an enzymatic portion to cut target RNA.

The term “PNA (peptide nucleic acid)” in the present invention means a molecule having the characteristics of both nucleic acid and protein, which is capable of complementarily binding to DNA or RNA. PNA was first reported in 1999 as similar DNA in which nucleobases are linked via a peptide bond (Nielsen P E, Egholm M, Berg R H, Buchardt O, “Sequence-selective recognition of DNA by strand displacement with a thymine-substituted polyamide”, Science 1991, Vol. 254: pp 1497-1500). PNA is absent in the natural world and artificially synthesized through a chemical method. PNA is reacted with a natural nucleic acid having a complementary base sequence through hybridization response, forming double strand. In the double strand with the same length, PNA/DNA and PNA/RNA double strand are more stable than DNA/DNA and DNA/RNA double strand, respectively. The form of repeating N-(2-aminoethyl)-glycine units linked by amide bonds is commonly used as a basic peptide backbone. In this context, the backbone of peptide nucleic acid is electrically neutral in comparison to that of natural nucleic acids having negative charge. Four bases of nucleic acid present in PNA are almost the same to those of natural nucleic acid in the respect of spatial size and distance between nucleobases. PNA has not only a chemical stability compared with natural nucleic acid, but also a biological stability due to no degradation by a nuclease or protease.

The term “antisense oligonucleotide” used herein is intended to refer to nucleic acids, preferably, DNA, RNA or its derivatives, that are complementary to the base sequences of a target mRNA, characterized in that they bind to the target mRNA and interfere its translation to protein. The antisense oligonucleotide of the present invention refers to DNA or RNA sequences which are complementary to a target mRNA, characterized in that they bind to the target mRNA and interfere its translation to protein, translocation into cytoplasm, maturation or essential activities to other biological functions. The length of antisense nucleic acids is in a range of 6-100 nucleotides and preferably 10-40 nucleotides.

The antisense oligonucleotides may be modified at above one or more positions of base, sugar or backbone to enhance their functions [De Mesmaeker, et al., Curr Opin Struct Biol., 5(3): 343-55 (1995)]. The oligonucleotide backbone may be modified with phosphothioate, phosphotriester, methyl phosphonate, single chain alkyl, cycloalkyl, single chain heteroatomic, heterocyclic bond between sugars, and so on. In addition, the antisense nucleic acids may include one or more substituted sugar moieties. The antisense oligonucleotides may include a modified base. The modified base includes hypoxanthine, 6-methyladenine, 5-me pyrimidine (particularly, 5-methylcytosine), 5-hydroxymethylcytosine (HMC), glycosyl HMC, gentobiosyl HMC, 2-aminoadenine, 2-thiouracil, 2-thiothymine, 5-bromouracil, 5-hydroxymethyluracil, 8-azaguanine, 7-deazaguanine, N6(6-aminohexyl)adenine, 2,6-diaminopurine, and so forth.

According to an embodiment, the mutation-inducing agent is a plant transformation vector which is inserted into a gDNA (genomic DNA) to target a protein consisting of the amino acid sequence of SEQ ID NO:2, and more preferably, T-DNA.

As used herein, the term “T-DNA” refers to a DNA fragment as a transfer DNA in Ti (tumor-inducing) plasmid of Agrobacterium sp., which is transferred into a nucleus of a host plant cell. A 25 bp repeat sequence is present in both termini of T-DNA, and DNA transfer proceeds at the direction from a left border to a right border.

A bacterial T-DNA with about 20,000 in length destroys a target gene by insertion, resulting in insertional muatagenesis. In addition to mutation, inserted T-DNA sequence may label a target gene. Insertion of T-DNA to a gene of interest may be induced by utilizing particular T-DNA selected from T-DNA collection (The SAIL collection). Since chromosome insertion site to each T-DNA collection has been known, it is possible to insert T-DNA into certain gene [Alonso, et al., Science, vol. 301, no. 5633, p. 653-657 (2003)].

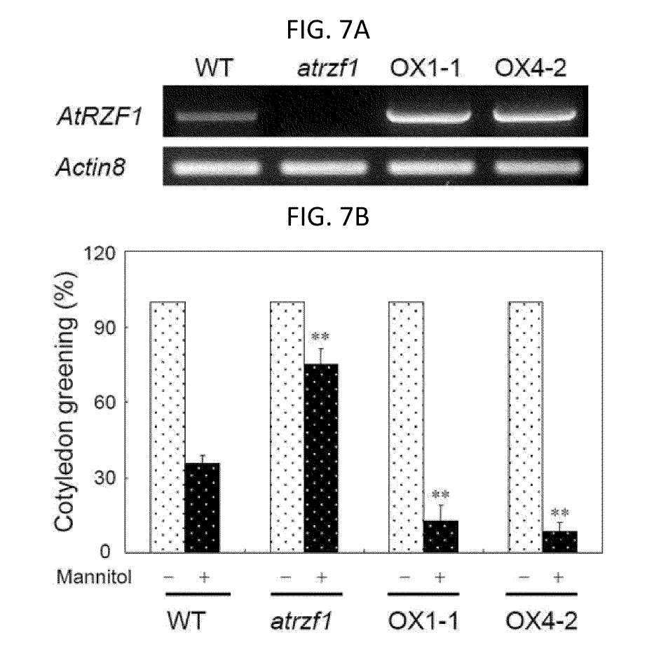

According to this invention, the present inventors have prepared mutant lines for suppressing the expression of AtRZF1 gene by means of Ti-plasmid transformation, and verified that T-DNA is inserted into exon 1 of AtRZF1 gene by a genotyping PCR using T-DNA border primer and gene-specific primers in front or back of T-DNA-inserted portion. In addition, the present inventors have demonstrated inhibition of AtRZF1 gene expression by RT-PCR method using RNA extracted from mutant lines (FIG. 7a).

In another aspect of this invention, there is provided a plant cell having drought stress tolerance, transformed with an inactivated E3 ubiquitin ligase atrzf1.

In still another aspect of this invention, there is provided a plant having drought stress tolerance, transformed with an E3 ubiquitin ligase atrzf1.

According to the present invention, the inactivation of E3 ubiquitin ligase atrzf1 may be carried out by inducing a mutation of atrzf1 gene. Preferably, the inactivation may be employed by insertion of T-DNA into a gDNA (genomic DNA) to target a protein consisting of the amino acid sequence of SEQ ID NO:2.

To introduce a foreign nucleotide sequence into plant cells or plants may be performed by the methods (Methods of Enzymology, Vol. 153, 1987) known to those skilled in the art. The plant may be transformed using the foreign nucleotide inserted into a carrier (e.g., vectors such as plasmid or virus) or Agrobacterium tumefaciens as a mediator [Chilton, et al., Cell, 11: 263-271 (1977)] and by directly inserting the foreign nucleotide into plant cells (Lorz, et al., Mol. Genet., 199: 178-182 (1985); the disclosure is herein incorporated by reference). For example, electroporation, microparticle bombardment, polyethylene glycol-mediated uptake may be used in the vector containing no T-DNA region.

Generally, Agrobacterium tumefaciens-mediated transformation is the most preferable for plant cells or seeds (U.S. Pat. Nos. 5,004,863, 5,349,124 and 5,416,011). The skilled artisan can incubate or culture the transformed cells or seeds to mature plants in appropriate conditions.

The term “plant(s)” as used herein, is understood by a meaning including a plant cell, a plant tissue and a plant seed as well as a mature plant.

The plants applicable of the present method include, but not limited to, most dicotyledonous plants including lettuce, chinese cabbage, potato and radish, and most monocotyledonous plants including rice plant, barley and banana tree, and especially, the present method may be effectively applied to enhance storage efficiency in edible vegetables or fruits such as tomato with thin pericarp, which represent rapid quality reduction depending on aging, and a plant of which leaves are traded as a major product. Preferably, the present method can be applied to the plants selected from the group consisting of food crops such as rice, wheat, barley, corn, bean, potato, Indian bean, oat and Indian millet; vegetable crops such as Arabidopsis sp., Chinese cabbage, radish, red pepper, strawberry, tomato, watermelon, cucumber, cabbage, melon, pumpkin, welsh onion, onion and carrot; crops for special use such as ginseng, tobacco, cotton, sesame, sugar cane, sugar beet, Perilla sp., peanut and rape; fruit trees such as apple tree, pear tree, jujube tree, peach tree, kiwi fruit tree, grape tree, citrus fruit tree, persimmon tree, plum tree, apricot tree and banana tree; flowering crops such as rose, gladiolus, gerbera, carnation, chrysanthemum, lily and tulip; and fodder crops such as ryegrass, red clover, orchardgrass, alfalfa, tallfescue and perennial ryograss.

In still another aspect of this invention, there is provided a method for preparing a transgenic plant with enhanced drought stress tolerance, comprising the steps of:

(a) inactivating an E3 ubiquitin ligase atrzf1 in a plant cell; and

(b) obtaining the transgenic plant with enhanced drought stress tolerance from the plant cell.

According to a preferable embodiment, the inactivation of E3 ubiquitin ligase atrzf1 is carried out using T-DNA insertion into an E3 ubiquitin ligase atrzf1 consisting of the nucleotide sequence of SEQ ID NO:3.

T-DNA is introduced into a plant cell as a form contained in the recombinant vector for plant transformation

According to a preferable embodiment, the recombinant vector for plant transformation is an Agrobacterium binary vector.

The term “binary vector” as used herein, refers to a cloning vector containing two separate vector systems harboring one plasmid responsible for migration consisting of left border (LB) and right border (RB), and the other plasmid for target gene-transferring. Any Agrobacterium suitable for expressing the nucleotide of this invention may be used, and most preferably, the transformation is carried out using Agrobacterium tumefaciens GV3101.

Introduction of the recombinant vector of this invention into Agrobacterium can be carried out by a large number of methods known to one skilled in the art. For example, particle bombardment, electroporation, transfection, lithium acetate method and heat shock method may be used. Preferably, the electroporation is used.

Selection of the transformed plant cell can be performed by exposing it to selective agents (e.g., metabolic inhibitors, antibiotics or herbicides). Transformed plant cells stably harboring marker genes which give a tolerance to selective agents are grown and divided in above culture. The exemplary markers include, but not limited to, hygromycin phosphotransferase (hpt), glyphosate-resistance gene and neomycin phophotransferase (nptII) system. The methods for developing or regenerating plants from plant protoplasms or various ex-plants are well known to those skilled in the art. The development or regeneration of plants containing the foreign gene of interest introduced by Agrobacterium may be achieved by methods well known in the art (U.S. Pat. Nos. 5,004,863, 5,349,124 and 5,416,011).

In further still another aspect of this invention, there is provided a method for conferring drought stress tolerance on a plant, comprising the steps of:

(a) inactivating an E3 ubiquitin ligase atrzf1 in a plant cell; and

(b) obtaining the transgenic plant with enhanced drought stress tolerance from the plant cell.

### ADVANTAGEOUS EFFECTS

The features and advantages of the present invention will be summarized as follows:

(a) the present invention provides a composition for improving drought stress tolerance in a plant, a transgenic plant with enhanced drought stress tolerance, and a method for preparing a transgenic plant.

(b) the novel functional plant having excellent drought stress-tolerance may be prepared using the composition for improving drought stress tolerance and a method for preparing a transgenic plant.

The present invention will now be described in further detail by examples. It would be obvious to those skilled in the art that these examples are intended to be more concretely illustrative and the scope of the present invention as set forth in the appended claims is not limited to or by the examples.

### Examples

### Materials and Methods

### Overexpression Construct of AtRZF1 (OX1-1 and OX4-2)

Total RNA was isolated from 2-week-old Arabidopsis leaves using Trizol reagent (Invitrogen, Carlsbad, Calif., USA). Reverse transcription (RT)-PCR was employed to obtain full-length AtRZF1 cDNA (At3g56580). The RT-PCR primers for gene amplification were ForXI 5′-TCTAGAATGTCAAGTATTCGGAATAC-3′ (SEQ ID NO: 4) (XbaI site underlined) and RevSI 5′-GTCGACATAGTCAAAAGGCCATCCAC-3′ (SEQ ID NO: 5) (SalI site underlined). Amplification proceeded for 35 cycles as follows: 94° C., 30 s; 55° C., 30 s; and 72° C., 1 min. The PCR-amplified products were cloned into the pGEM T-easy vector and then the sequence of AtRZF 1 cDNA was confirmed by DNA sequencing analysis. Afterwards, the products were double digested with XbaI and SalI and directionally cloned into the plant expression vector pBI121-1. The resultant construct was then introduced into Agrobacterim tumefaciens strain GV3101 via in planta vacuum infiltration (Bechtold and Pelletier, 1998). Homozygous lines (T₃ generation) from 12 independent transformants were obtained, and two lines (OX1-1 and OX4-2) with high transgene expression levels were selected for phenotypic characterization related to drought stress. Kanamycin resistance of the T₂ generation from these two selected lines was segregated as a single locus.

### Homozygous Artzf1 Mutant Lines

The AtRZF1 T-DNA insertion line SALK_{—}024296 (atrzf1) was acquired from the Arabidopsis T-DNA insertion collection of the Salk Institute (Alonso et al., 2003). To select plants homozygous for the T-DNA insertion, gene-specific primers (forward, 5′-TCTAGAATGTCAAGTATTCGGAATAC-3′ (SEQ ID NO: 6); and reverse, 5′-GTCGACATAGTCAAAAGGCCATCCAC-3′) (SEQ ID NO: 7) were utilized for the atrzf1 line. Genomic DNA was extracted from atrzf1 mutant and WT (ecotype, Col-0) lines and subjected to PCR analysis using the gene-specific primers. As a result, PCR-amplified products for AtRZF1 were obtained in WT, but not in the atrzf1 line. Subsequently, the presence of the T-DNA insertion was confirmed by using the gene-specific forward primer in combination with the T-DNA left border specific primer 5′-GCGTGGACCGCTTGCTGCACCT-3′ (SEQ ID NO: 8). It has been verified that T-DNA was inserted into exon 1 of AtRZF1. To obtain excellent homozygous atrzf1 mutant lines, Amplification proceeded for 35 cycles as follows: 94° C., 30 s; 57° C., 30 s; and 72° C., 1 min. To assess transcription of AtRZF1 in the atrzf1 mutant line, RT-PCR was carried out. After germination, total RNA was isolated from 10-day-old atrzf1 mutant seedling using Trizol reagent (Invitrogen, Carlsbad, Calif., USA). For cDNA synthesis, total RNA (5 μg), oligo-(dT)₁₅ primer and SuperScript II reverse transcriptase (Invirogen, Gaithersburg, Md., USA) were used. PCR amplification using the synthesized cDNA and AtRZF1 gene-specific primers was carried out for 27 cycles as follows: 94° C., 30 s; 55° C., 30 s; and 72° C., 1 min. As a result, no AtRZF1 transcripts were detected in atrzf1 mutant lines, suggesting that an atrzf1 mutant line is an AtRZF1 knock-out plant. Arabidopsis Actin8 served as a control for RT-PCR analysis. The primer set for Actin8 is as follows: forward primer, 5′-CCTTGCTGGTCGTGACCTTACTGA-3′ (SEQ ID NO: 9); and reverse primer, 5′-CTCTCAGCACCGATCGTGATCACT-3′ (SEQ ID NO: 10). PCR amplification for Actin8 was employed for 24 cycles as follows: 94° C., 30 s; 55° C., 30 s; and 72° C., 1 min.

### Growth Conditions and Stress Inductions

The plants were challenged with osmotic stress via the submerging of 2-week-old Arabidopsis seedlings in a solution containing 400 mM mannitol. Samples were obtained at 0, 6, 12, and 24 h after osmotic stress.

For drought stress, seedling plants were grown in pots with normal watering every 3 days. After 2 weeks, the plants were divided into two groups for stress treatments. One group was subjected to drought stress by withholding water for 10 days, and a control group was watered normally.

In each case, the retrieved seedlings were promptly frozen in liquid nitrogen and stored at −80° C.

### Localization of AtRZF 1-EGFP Fusion Proteins in Arabidopsis Protoplast Cells

For transient expression of the AtRZF1-GFP construct, the open reading frame of AtRZF1 was amplified with primers 5′-TCTAGAATGTCAAGTATTCGGAATAC-3′ (SEQ ID NO: 11) and 5′-CCCTTGCTCACCATATAGTCAAAAGGC-3′ (SEQ ID NO: 12), and EGFP gene sequences were amplified with primers 5′-GCCTTTTGACTATatggtgagcaaggg-3′ (SEQ ID NO: 13) and 5′-GAGCTCAGTTATCTAGATCC-3′ (SEQ ID NO: 14). The two PCR products were annealed, reamplified with primers 5′-TCTAGAATGTCAAGTATTCGGAATAC-3′ (SEQ ID NO: 15) and 5′-GAGCTCAGTTATCTAGATCC-3′ (SEQ ID NO: 16), and inserted into pGEM T-easy vector for DNA sequencing analysis. The construct was then digested with XbaI and NotI restriction enzymes, after which the fragment was cloned into the pB1221. The AtRZF1-EGFP fusion genes were transformed into Arabidopsis protoplast cells by means of polyethylene glycol (PEG) treatment (Abel and Theologis, 1994). The expression of AtRZF 1-EGFP and EGFP was monitored 12 h after transformation. Transformed protoplasts were placed on the slide glass and observed using a FluoView1000 confocal microscope (Olympus, Tokyo, Japan). Confocal images were obtained and processed using FV 10-ASW 1.7A software (Olympus).

Expression of Genes Associated with Drought Stress Using a Quantitative Real-Time PCR (qPCR)

Quantitative real-time PCR (qPCR) was carried out with a Rotor-Gene 6000 quantitative PCR apparatus (Corbett Research, Mortlake, NSW, Australia), and the results were analyzed using RG6000 1.7 software (Corbett Research). Total RNA was extracted from the dehydration stress-treated 14-day-old Arabidopsis seedlings using a RNeasy Plant Mini kit (Qiagen, Valencia, Calif., USA), of which 100 ng in each sample was subjected to qPCR using the SensiMix One-Step kit (Quantance, London, UK). The qPCR procedure is as follows: (a) 1^{st} PCR was performed for first, 42° C., 30 min; second, 95° C., 10 min; third, 95° C., 15 s; fourth, 55° C., 30 s; and 72° C., 30 s; and (b) 2^{nd} PCR was carried out for 35 cycles consisting of 95° C., 15 s; 55° C., 30 s; and 72° C., 30 s per one cycle.

Gene-specific primers for qPCR are shown in Table 1.

TABLE 1

| Gene | Primer sequence (5′ to 3′) | |
|---|---|---|
| AtRZF1 (At3g56580) | Forward: CAGAAGCACCAATGGAAGAG | (SEQ ID NO: 17) |
| | Reverse: GTCGACATAGTCAAAAGGCCATCCAC | (SEQ ID NO: 18) |
| P5CS1 (At2g39800) | Forward: CGACGGAGACAATGGAATTGT | (SEQ ID NO: 19) |
| | Reverse: GATCAGAAATGTGTAGGTAGC | (SEQ ID NO: 20) |
| P5CR (At5g14800) | Forward: GATGGAGATTCTTCCGATTCC | (SEQ ID NO: 21) |
| | Reverse: CCAGCTGCAACAGAAACCAGA | (SEQ ID NO: 22) |
| RAB18 (At5g66400) | Forward: CGATCCAGCAGCAGTATGAC | (SEQ ID NO: 23) |
| | Reverse: TTCGAAGCTTAACGGCCACC | (SEQ ID NO: 24) |
| RD29A (At5g52310) | Forward: GACGGGATTTGACGGAGAAC | (SEQ ID NO: 25) |
| | Reverse: CCGCCACATAATCTCTACCC | (SEQ ID NO: 26) |
| RD29B (At5g52300) | Forward: CGTCCTTATGGTCATGAGC | (SEQ ID NO: 27) |
| | Reverse: GCCTCATGTCCGTAAGAGG | (SEQ ID NO: 28) |
| AOX1a (At3g22370) | Forward: GGGTATCATTGATTCGATTA | (SEQ ID NO: 29) |
| | Reverse: GTTATGATGATATCAATGGT | (SEQ ID NO: 30) |
| COR15A (At2g42540) | Forward: CAGCGGAGCCAAGCAGAGCAG | (SEQ ID NO: 31) |
| | Reverse: CATCGAGGATGTTGCCGTCACC | (SEQ ID NO: 32) |
| ERD15 (At2g41430) | Forward: CCAGCGAAATGGGGAAACCA | (SEQ ID NO: 33) |
| | Reverse: ACAAAGGTACAGTGGTGGC | (SEQ ID NO: 34) |
| ERD1 (At5g51070) | Forward: GTAAGGTCATTCTCTTCATAGATG | (SEQ ID NO: 35) |
| | Reverse: CTGAAGTTCACCCCTTCCAAGTG | (SEQ ID NO: 36) |
| Actin8 (At1g49240) | Forward: CCTTGCTGGTCGTGACCTTACTGA | (SEQ ID NO: 37) |
| | Reverse: CTCTCAGCACCGATCGTGATCACT | (SEQ ID NO: 38) |

### In Vitro Self-Ubiqutination Assay

Full-length AtRZF1 cDNA was amplified using the following primers: 5′-GAATTCATGTCAAGTATTCGGAATAC-3′ (SEQ ID NO: 39) (EcoRI site underlined) and 5′-GTCGACATAGTCAAAAGGCCATCCAC-3′ (SEQ ID NO: 40) (SalI site underlined). PCR products were cleaved with EcoRI and SalI and inserted into a pMAL P2x vector (New England BioLabs, Beverly, Mass., USA). This plasmid was expressed in E. coli strain BL21 and purified by affinity chromatography using amylase resin (New England BioLabs). In vitro self-ubiqutination assay was carried out with Auto-ubiquitinylation kit (Enzo Life Sciences, Farmingdale, N.Y., USA).

### Immunoblot Analysis

Reaction samples were separated by 12% sodium dodecyl sulfate-polyacrylamide gel electrophoresis (SDS-PAGE) and transferred to polyvinylidene fluoride (PVDF) membranes (Millipore, Bedford, Mass., USA) using a semi-dry transfer cell (Bio-Rad, Hercules, Calif., USA). The membranes was blocked with weak shaking at room temperature for 2 h using BSA/PBS-T buffer [PBS (phosphate-buffered saline) solution (137 mM NaCl, 3 mM KCl, 10 mM Na₂HPO₄, 2 mM KH₂PO₄; pH7.4) supplemented with 1% BSA (bovine serum albumin) and 0.2% tween-20]. The blocked membrane was washed 3 times with PBS-T buffer [PBS solution (137 mM NaCl, 3 mM KCl, 10 mM Na₂HPO₄, 2 mM KH₂PO₄; pH7.4) supplemented with 0.2% tween-20] at room temperature for 10 min, and then incubated with BSA/PBS-T buffer supplemented with primary anti-MBP (Maltose Binding Protein) antibody (1:10,000 dilution; New England BioLabs) or primary anti-Ub antibody (1:500 dilution; Enzo Life Sciences) at 4° C. for 12 h with weak shaking. The blocked membrane was then washed 3 times with PBS-T buffer at room temperature for 10 min, and incubated with BSA/PBS-T buffer supplemented with secondary anti-mouse antibody-peroxidase conjugates (1:10,000 dilution; New England BioLabs) or secondary anti-rabbit IgG antibody-HRP conjugates (1:5,000 dilution; Enzo Life Sciences) at room temperature for 2 h with weak shaking. Finally, the membrane was washed 6 times with PBS-T at room temperature for 10 min, and exposed on a X-ray film using Supersignal West Pico ECL Substrate kit (Thermo Scientific).

### Results

Identification and Amino Acid Sequence Analysis of the AtRZF1 (At3g56580) Gene

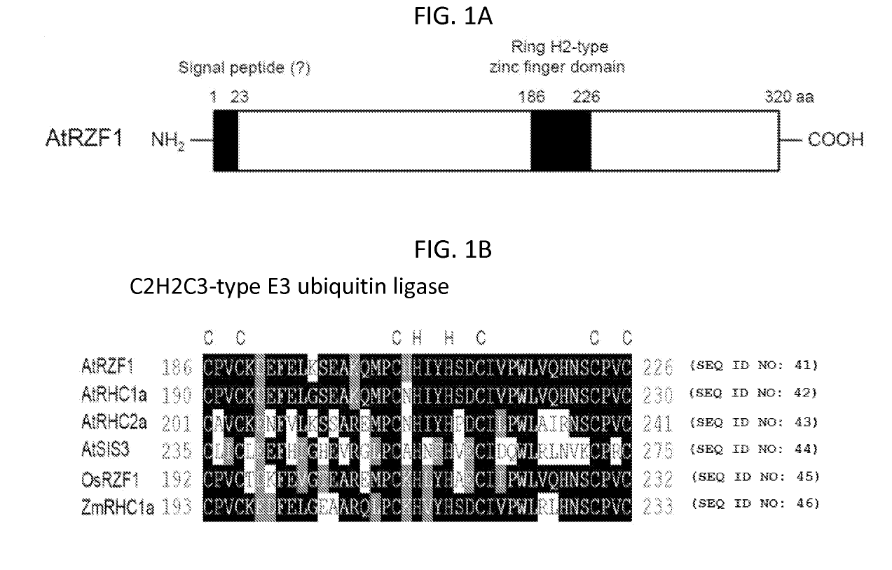

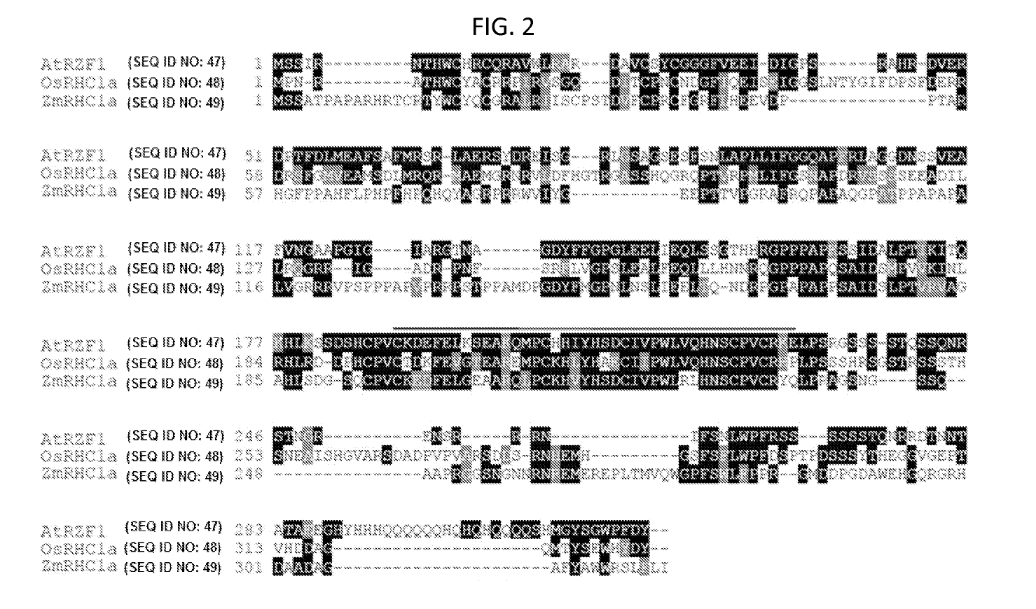

The present analysis determined that At3g56580 belongs to the C3H2C3-type RING-H2 finger gene family in the complete Arabidopsis genome sequence analysis (Stone et al., 2005). At3g56580 was comprised of 963 bp and harbored one single open reading frame encoding a 320 amino acid protein with a calculated molecular weight of 35.8 kDa. The protein harbored a predicted signal peptide sequence as shown by several software programs (http://ihg.gsf.de; http://hannibal.biol.uoa.gr) (FIG. 1A). As shown in FIG. 1B, the deduced amino acid sequence displayed considerable homology with known members of the RING-H2 Zinc Finger protein family. The A. thaliana At3g56580 gene was therefore designated as AtRZF 1. An amino acid sequence alignment between AtRZF1 and unknown protein orthologs from rice and maize is also shown in FIG. 2. Overall homology values of 36-70% identity and 43-75% similarity were observed between AtRZF1 and the orthologous proteins from rice and maize.

AtRZF1 contains a single RING domain in its central region that is 39-95% identical to the corresponding region of Arabidopsis and other plant RING proteins. Functions of these proteins are also unknown except for a Sugar-insensitive 3 (SIS3). SIS3 encodes an ubiquitin E3 ligase that is a positive regulator of sugar signaling during early seedling development (Huang et al., 2010). Because the Cys_{X2}-Cys-_{X14}-Cys-_{X1}-His-_{X2}-His-_{X2}-Cys-_{X10}-Cys-_{X2}-Cys sequence is well conserved in the 41 amino acid RING motif (FIG. 1B), AtRZF1 is a C3H2C3-type RING-H2 protein (Jensen et al., 2005).

### Subcellular Localization of AtRZF1 Protein

Several software programs were used to examine the AtRZF1 amino acid sequence for predicted domains (FIG. 1). The Mitoprot2 (http://ihg.gsf.de) and PredSL (http://hannibal.biol.uoa.gr) program predicted that AtRZF1 has a mitochondria signal peptide from amino acids 1 to 23. In contrast, the PrediSi program (http://www.predisi.de) predicted that AtRZF1 does not have a signal peptide. Thus, it is unclear at this point whether AtRZF 1 has a signal peptide or not.

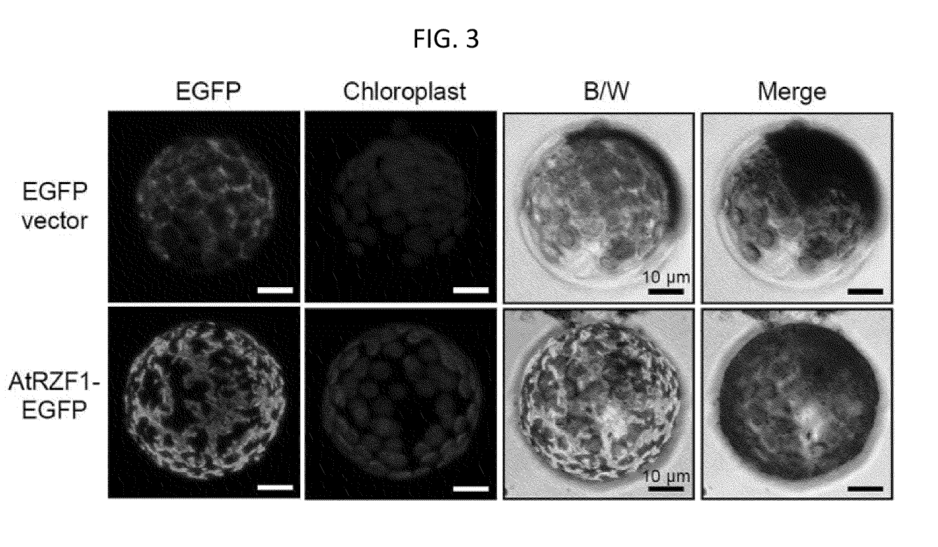

To determine the subcellular localization of AtRZF1, a green fluorescent protein (GFP) reporter gene was fused in-frame to the AtRZF1 coding region to generate an AtRZF1-EGFP fusion protein in Arabidopsis protoplast cells using a PEG-mediated method. As shown in FIG. 3, the epifluorescence signal of the AtRZF1-EGFP construct was detected in the cytoplasm of the Arabidopsis mesophyll protoplast cells. These results demonstrated that the subcellular localization of AtRZF1 is the same as that of EGFP vector as a cytoplasm-localized protein.

### AtRZF1 Expression in Arabidopsis

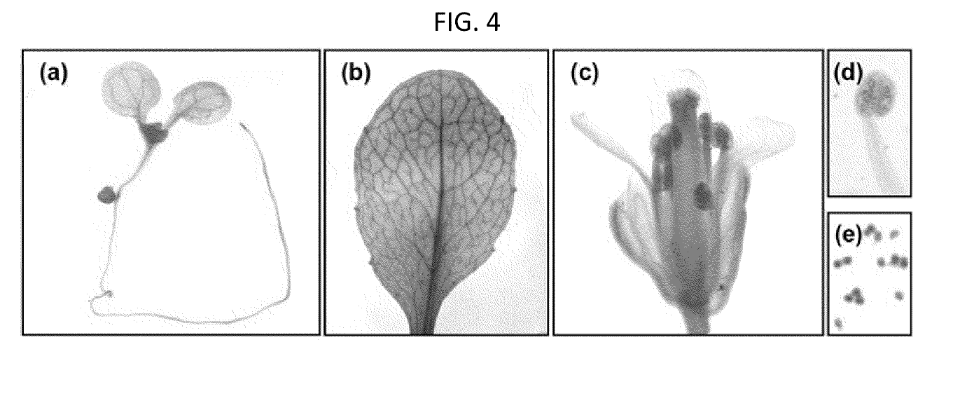

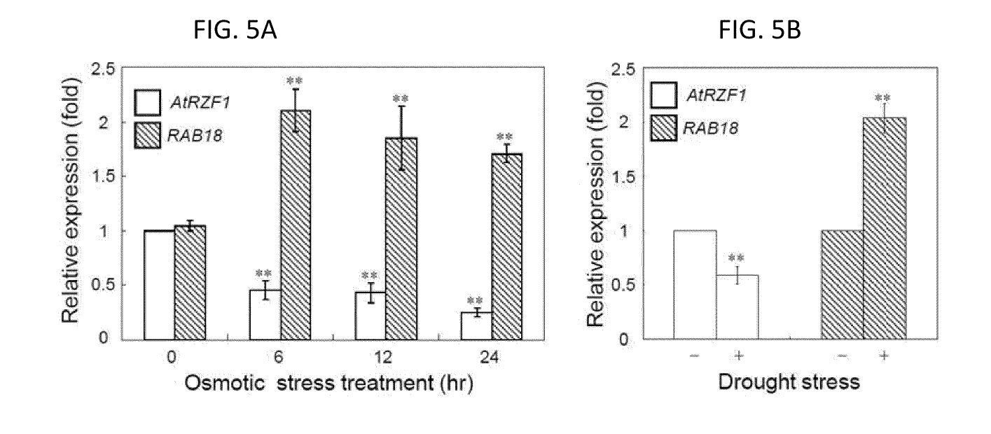

To obtain clues regarding the functions of AtRZF1, its expression pattern was initially assessed by histochemical GUS staining of Arabidopsis transgenic plants harboring the 1.146-kb AtRZF1 promoter-GUS fusion construct. Analysis of the transgenic plants revealed strong GUS activity in the whole seedling plant and, especially, in the vascular system ((a) in FIG. 4 and (b) in FIG. 4). In flowers, GUS staining was observed in the sepal ((c) in FIG. 4), anther of stamen ((d) in FIG. 4), and the pollens ((e) in FIG. 4). Genome-wide expression analysis in Arabidopsis revealed that the expression of AtRZF1 was reduced by osmotic stress (http://jsp.weigelworld.org). Next, in an effort to determine the in vivo functions of AtRZF1, the present inventors assessed the accumulation of AtRZF1 mRNA in 2-week-old Arabidopsis seedlings during osmotic stress using quantitative real-time PCR. As shown in FIG. 5A, the transcript levels of AtRZF1 were reduced by as much as 3-fold after 24 h of mannitol treatment. Drought treatment also produced a reduction of AtRZF1 expression in Arabidopsis leaves (FIG. 5B). The osmotic stress-inducible Responsive to ABA 18 (RAB18) (Huang et al., 2008) gene was used as a control for the water deficit stress treatment (FIG. 5A and FIG. 5B). These results strongly suggest that AtRZF1 is regulated by dehydration condition.

### AtRZF1 Exhibits In Vitro Ubiquitin E3 Ligase Activity

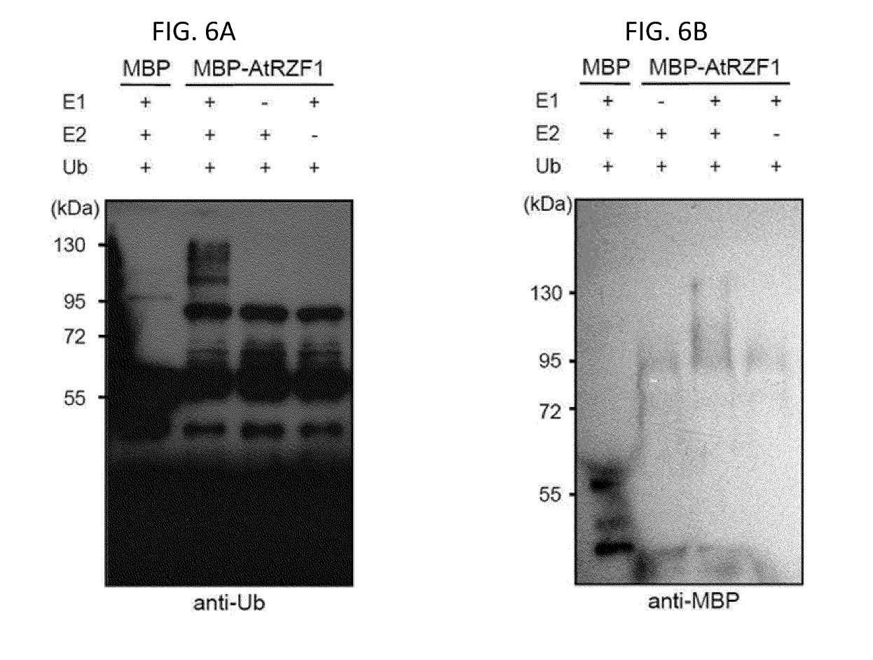

RING domain proteins are one type of E3 ligase involved in the ubiquitination process (Lorick et al., 1999). The AtRZF1 protein is a member of the C3H2C3-type RING-H2 protein (FIG. 1B) and has not previously been tested for E3 ligase activity (Kim et al., 2012). Thus, it was of interest to test the ability of AtRZF1 to function as an E3 ligase in the ubiquitination process. Toward this end, AtRZF1 was tested for E3 ligase activity using in vitro assays (FIGS. 6A and 6B). Recombinant MBP-AtRZF1 protein was produced in Escherichia coli and affinity purified using amylase resin. In the presence of E1 and E2, ubiquitinated MBP-AtRZF1 proteins were detected by immunoblot analysis using anti-Ub (FIG. 6A) and anti-MBP (FIG. 6B) antibodies. In the absence of either E1 or E2, the ubiquitination activity was not observed with MBP-AtRZF1. As shown in FIGS. 6A and 6B, high-molecular-mass ubiquitinated bands were produced by AtRZF1, indicating that AtRZF1 had Ub E3 ligase activity in vitro.

### Overexpression of AtRZF1 Confers High Sensitivity to Drought Stress

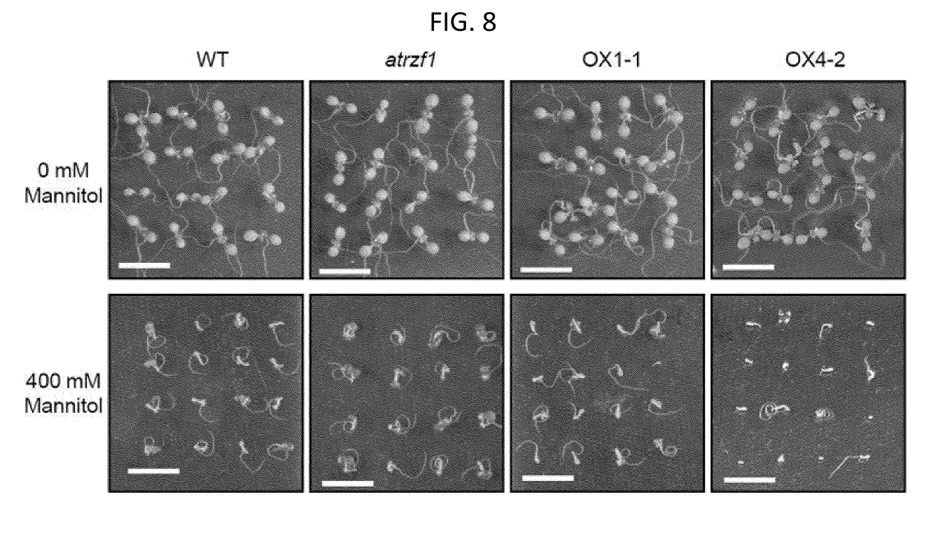

To investigate the in vivo function of AtRZF1, AtRZF1 overexpression was induced in Arabidopsis under the control of the 35S promoter. Twelve homozygous lines (T₃ generation) were obtained, and two lines (OX1-1 and OX4-2) exhibiting high levels of transgene expression (FIG. 7a) were selected for phenotypic characterization. The comparison of AtRZF1-overexpressing lines with WT plants demonstrated no morphological alterations or retardation of growth (FIG. 8). In an effort to further evaluate the function of AtRZF 1 in Arabidopsis, the present inventors obtained the At3g56580-tagged T-DNA insertion mutant SALK_{—}024296. In addition, the mutant line was prepared by T-DNA insertion into exon 1 of the At3g56580 gene, leading to inhibit endogenous expression of AtRZF1 gene. The T-DNA inserted in exon 1 of the At3g56580 gene was verified by PCR and the cloning of the left T-DNA border. Once homozygosity had been established, the absence of AtRZF1 was verified via RT-PCR (FIG. 7a).

To evaluate the effects of AtRZF1 expression on germination with elevated mannitol, the seeds of the WT, atrzf1, and AtRZF1-overexpressing plants were germinated in MS media supplemented with 400 mM mannitol, then permitted to grow for 8 days prior to assessment of the survival rates of the AtRZF1-overexpressing plants in response to dehydration stress (FIG. 8). At 400 mM mannitol, approximately 35% of the WT leaves expanded and turned green, as compared to less than 15% of the OX1-1 and OX4-2 lines (FIG. 7b). On the contrary, 80% of atrzf1 mutant line remained alive at 8 days after germination (FIG. 7b). Thus, AtRZF1-overexpressing plants were hypersensitive to osmotic stress in terms of cotyledon development, demonstrating that the atrzf1 mutant and AtRZF1-overexpressing plants had the opposite phenotype in response to drought stress.

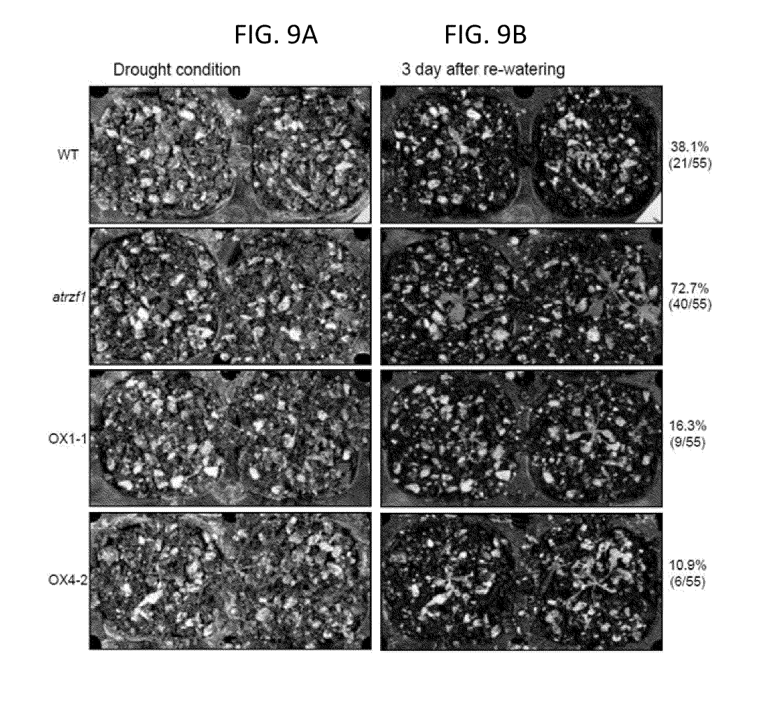

Next, the present inventors investigated the capacity of atrzf1 mutant plant to respond to severe drought stress. To further evaluate the responses to drought stress, WT, atrzf1, and AtRZF1-overexpressing plants were grown for 2 weeks in pots under normal growth conditions and further grown for 10 days without watering to completely dry the soil. These plants were re-watered and their survival rates were determined. As shown in FIGS. 9A and 9B, most WT and AtRZF1-overexpressing plants were seriously wilted and impaired (FIG. 9A) and, after re-watering for 3 days, the survival rates were 38.1% (21 of 55) and 16.3% (nine of 55) for line OX1-1) to 10.9% (six of 55) for line OX4-2 (FIG. 9B). These results indicate that gain-of-function transgenic plants were more susceptible to water deficit than WT plants. On the other hand, atrzf1 mutant appeared relatively healthy after this severe drought condition (FIG. 9A). After 3 days of re-watering, the survival rate of atrzf1 mutant was 72.7% (40 of 55) (FIG. 9B). This strongly suggested that atrzf1 mutant plant, as opposed to AtRZF1-overexpressing transgenic plants, was highly tolerant to severe water stress.

### Sensitivity to Drought AtRZF 1-Overexpressing Plants

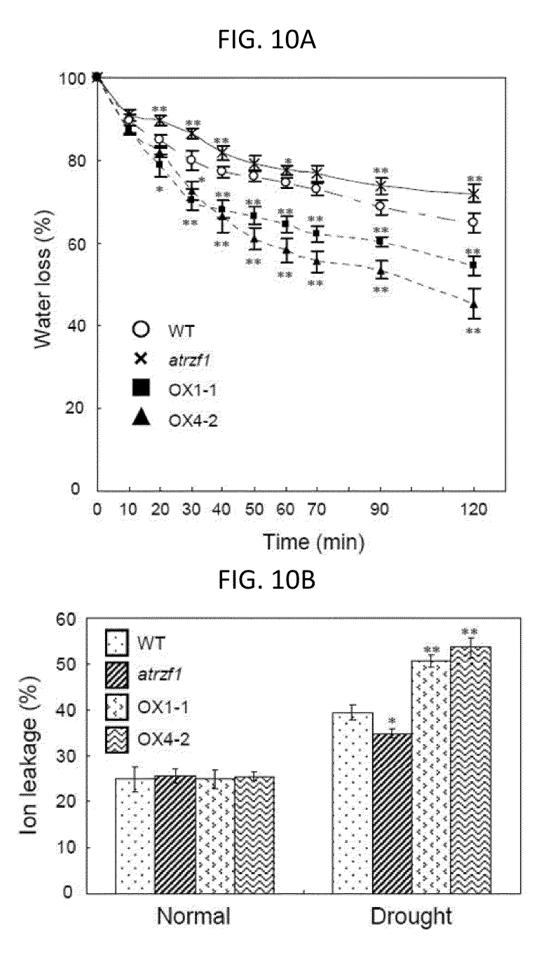

To further evaluate the responses to drought stress, cut rosette water loss rates (CRWL) of the plants were estimated. To assess water loss from leaves, leaves of similar size, age, and positions on WT, atrzf1, and AtRZF1-overexpressing plants were detached and measured for decreases in fresh weight, as described previously (Sang et al., 2001). After detachment, leaves from the AtRZF1-overexpressing plants exhibited higher loss of fresh weight than those from WT and atrzf1 plants under ambient conditions (FIG. 10A). The difference became more apparent with the lapse of time following detachments. Drought stress also involves the disruption of plasma membrane integrity as the final step in cell death. This can be conveniently quantified by electrolyte leakage (Nanjo et al., 1999). The drought-induced phenotype was delayed in the atrzf1 line, as shown by lowered membrane ion leakage of the leaves compared with WT and AtRZF1-overexpressing plants (FIG. 10B). These results indicate that physiological processes of drought-induced phenotype began earlier in the AtRZF1-overexpressing line than in the WT and atrzf1 plants.

### Effects of Drought on Stress-Related Genes

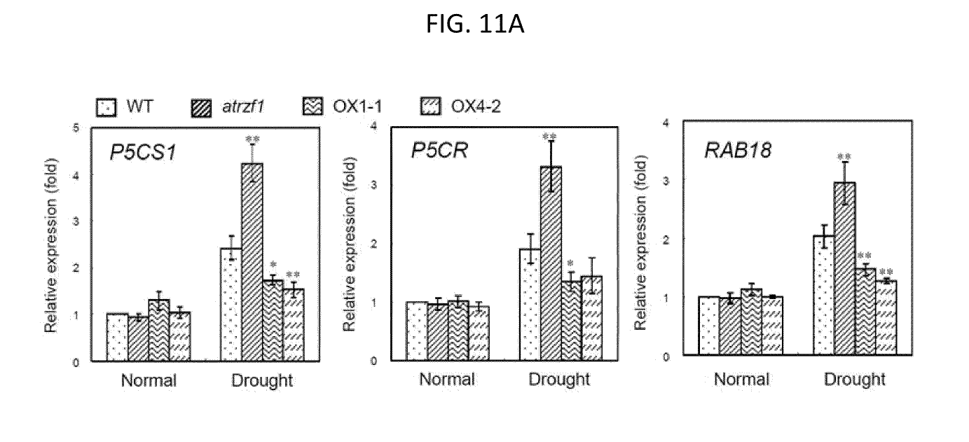

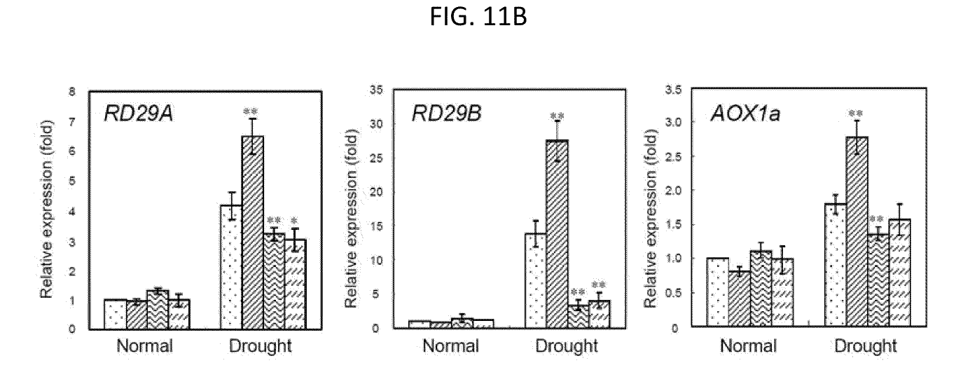

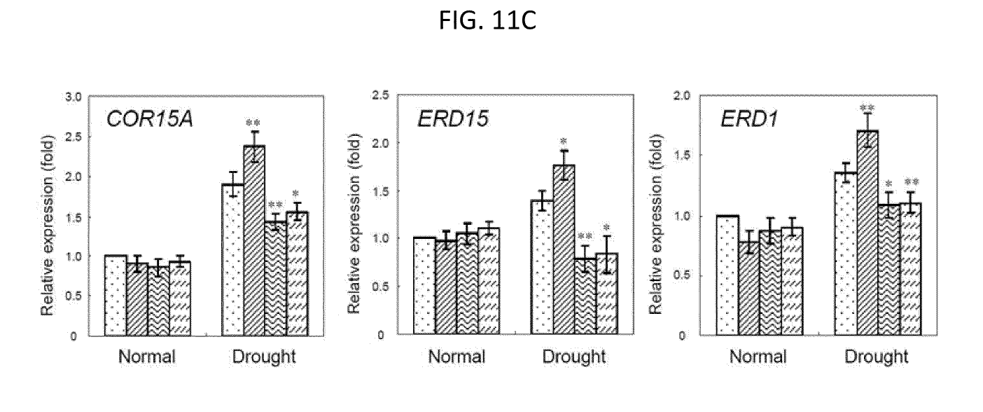

The expressions of the Delta1-Pyrroline-5-Carboxylate Synthase 1 (P5CS1), Delta1-Pyrroline-5-Carboxylate Reductase (P5CR), RAB18, Responsive to Dessication 29A (RD29A), RD29B, Alternative Oxidase 1a (AOX1a), COLD-REGULATED 15A (COR15A), EARLY RESPONSIVE TO DEHYDRATION 15 (ERD15), and ERD1 genes are induced by stress (Savouré et al., 1997; Strizhov et al., 1997; Tran et al., 2006; Vanlerberghe et al., 2009; Lim et al. 2010). In detail, P5CS1, COR15A, and ERD1 genes are induced by salt and drought stress conditions. FIGS. 11A through 11C show that the transcript levels of stress-inducible genes including P5CS1, P5CR, RAB18, RD29A, RD29B, AOX1a, COR15A, ERD15, and ERD1 displayed reduced induction in AtRZF1-overexpressing OX1-1 and OX4-2 lines than in the WT and atrzf1 plants following drought treatment. However, transcription of the nine genes was more induced by drought treatment in the atrzf1 mutant lines than in the WT plants. Taken together, our expression data suggest that AtRZF1 acts negatively on drought stress-related genes.

### Higher Proline Content of Atrzf1 Mutant Under Drought Stress

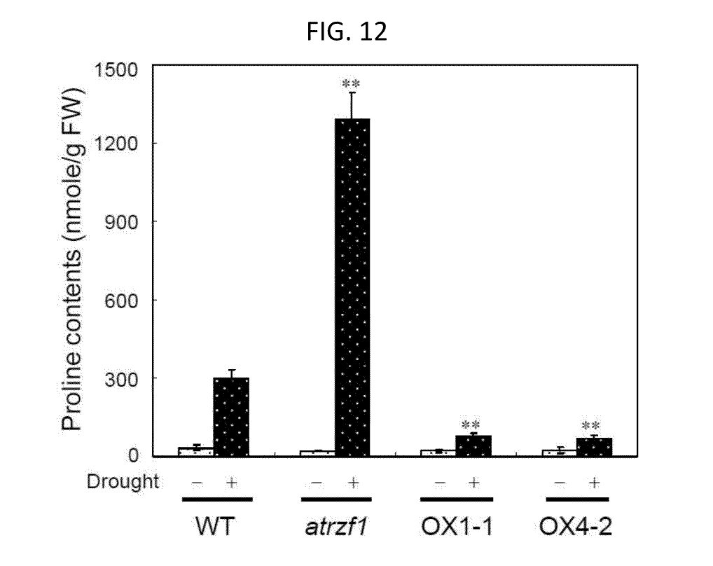

Because the transcripts of P5CS1 and P5CR genes increased in atrzf1 mutant line, the present inventors determined the proline content in rosette leaves of WT and transgenic plants. To assess whether there were differences in the accumulation of proline among WT, atrzf1, and AtRZF1-overexpressing plants, the proline contents of leaves were determined at 10 days after drought treatment. Before stress, the contents of proline were at similarly low levels in all seedlings (FIG. 12). Under drought stress, a significant difference in proline content was observed among WT, atrzf1-, and AtRZF1-overexpressing plants. With regard to proline content, the atrzf1 mutant exhibited higher levels than the WT and AtRZF1-overexpressing plants. The content of proline was much more induced by drought treatment in WT plants than in the AtRZF1-overexpressing plants (FIG. 12). These results suggested that AtRZF1 participates negatively in proline production under drought condition.

### Discussion

The present inventors demonstrate that the AtRZF1 gene, which encodes a deduced C3H2C3-type RING zinc finger protein, plays an important role in drought response. Water deficit response assays indicated that, while the atrzf1 mutant was less sensitive to drought, AtRZF1-overexpressing plants were more sensitive, suggesting that AtRZF1 negatively regulates the drought response during seed germination and early seedling development. Consequently, the present invention demonstrates a distinct difference in water loss and ion leakage between AtRZF1-overexpressing transgenic and atrzf1 mutant plants (FIG. 10A). The leaves of AtRZF1-overexpressing lines exhibited a significant increase in water loss and in membrane ion leakage under drought condition compared with WT and atrzf1 mutant leaves (FIG. 10B).

The transcript levels of stress-inducible genes including P5CS1, P5CR, RAB18, RD29A, RD29B, AOX1a, COR15A, ERD15, and ERD1 displayed reduced induction in AtRZF1-overexpressing lines than in WT and atrzf1 plants following drought treatment (FIGS. 11A-11C). These results further prove that AtRZF1 acts negatively on drought stress responses. The atrzf1 mutant displayed significant drought tolerance when compared with WT and AtRZF1-overexpressing plants. The accumulation of proline in plant cells can protect the cells from osmotic stress (Szabados and Savouré, 2010). The accumulation of proline in atrzf1 was greater than that in WT and AtRZF1-overexpressing plants (FIG. 12), which might suggest that AtRZF1 is a component responsible for induction of leaf drought sensitivity through the modulation of osmolytic components. AtRZF 1 is annotated as a RING finger protein. As some RING finger proteins have been shown to play key roles in the ubiquitination/proteasome process by acting as ubiquitin E3 ligases (Smalle and Vierstra, 2004), AtRZF1 was tested for E3 ubiquitin ligase activity using an in vitro assay. Our analyses demonstrate that the AtRZF 1 protein is indeed an active E3 ligase based on the occurrence of autoubiquitination of the MBP-AtRZF1 fusion protein in the presence of the E1 and E2 enzymes (FIG. 6). Interestingly, many E3 ubiquitin ligases act as negative regulators of stress responses. For example, High Expression of Osmotically Responsive Genes 1 (HOS1) negatively regulates the expression of cold-responsive genes by ubiquitinating Inducer of CBP Expression 1 (ICE1) (Chinnusamy et al., 2007), and DREB2A-Interacting Protein 1 (DRIP1) targets ubiquitination of Dehydration-Responsive Element Binding protein 2A (DREB2A), resulting in its destabilization and down regulation of stress responses (Qin et al., 2008). While Carboxyl terminus of HSC70-Interacting Protein (AtCHIP) is reported to monoubiquitinate the A subunit of Protein Phosphatase 2A (PP2A) and increase its activity, AtCHIP-overexpressing Arabidopsis plants showed increased sensitivity to cold stress (Yan et al., 2003). A novel E3 ligase, Keep on Going (KEG) protein was recently found to be regulated by ABA (Stone et al., 2006). KEG interacts with and degrades ABA Insensitive 5 (ABI5), a positive regulator of ABA signaling. ABA promotes KEG degradation by self-ubiquitination and maintains a balance between KEG and ABI5. In addition, several E3 ligases have been shown to act as positive regulators of abiotic stress. These proteins include RING-H2Finger A2a (RHA2a) (Bu et al., 2009) and ABA Insensitive RING Protein 1 (AtAIRP1) (Ryu et al., 2010), which are involved in several aspects of ABA signaling. Proteins targeted by RHA2a, are proposed to be negative regulators of the ABA signaling pathway. Overexpression of AtAIPR1 leads to ABA hypersensitivity during seed germination and stomatal closure, resulting in tolerance to drought stress.

Based on our results, the present inventors hypothesize that AtRZF 1 functions as an E3 ligase that mediates the degradation of its substrates (yet to be identified) through the ubquitin-proteasome machinery. Given that AtRZF 1 itself negatively regulates drought response, the present inventors propose that the degraded proteins by AtRZF1 are positive regulators of water deficit stress and that removing these molecules has the effect of activating drought response. Thus, further functional studies of AtRZF1, its target proteins, and their interplay are necessary for complete understanding of the drought response networks in plants.

### REFERENCES

1. Abel S, Theologis A. 1994. Plant Journal 5: 421-427.
2. Alonso JMStepanova ANLeisse T J, Kim C J, Chen H, Shinn P. et al. 2003. Genome-wide insertional mutagenesis of Science 301: 653657
3. Bu Q, Li H, Zhao Q, Jiang H, Zhai Q, Zhang J, Wu X, Sun J, Xie Q, Wang D, Li C. 2009. Plant Physiology 150:463-481.
4. Chen Z J, Sun L J. 2009. Molecular Cell 33:275-286.
5. Chinnusamy V, Zhu J, Zhu J K. 2007. Trends in Plant Science 12:444-451.
6. Craig A, Ewan R, Mesmar J, Gudipati V, Sadanandom A. 2009. Journal of Experimental Botany 60:1123-1132.
7. Dye B T, Schulman B A. 2007. Annual Review of Biophysics and Biomolecular Structure 36:131-150.
8. Freemont P S. 2000. Current Biology 10:R84-R87.
9. Freemont P S, Hanson I M, Trowsdale J. 1991. Cell 64:483-484.
10. Fujita Y, Fujita M, Shinozaki K, Yamaguchi-Shinozaki K. 2011. Journal of Plant Research 124:509-525.
11. Gmachl M, Gieffers C, Podtelejnikov A V, Mann M, Peters J M. 2000. Proceedings of the National Academy of Sciences, USA 97:8973-8978.
12. Harb A, Krishnan A, Ambavaram M M R, Pereira A. 2010. Plant Physiology 154:1254-1271.
13. Huang D, Wu W, Abrams S R, Cutler A J. 2008. Journal of Experimental Botany 59:2991-3007.
14. Huang Y, Li C Y, Pattison D L, Gray W M, Park S, Gibson S I. 2010. Plant Physiology 152:1889-1900.
15. Jakab G, Ton J, Flors V, Zimmerli L, Metraux J.P, Mauch-Mani B. 2005. Plant Physiology 139:264-274.
16. Jensen R B, Jensen K L, Jespersen H M, Shiver K. 1998. FEBS Letters 436:283-287.
17. Kim S J, Ryu M Y, Kim W T. 2012. Biochemical and Biophysical Research Communications 420:141-147.
18. Lim G H, Zhang X, Chung M S, Lee D J, Woo Y M, Cheong H S, Kim C S. 2010. New Phytologist 185:103-113.
19. Lin H K, Wang L, Hu Y C, Altuwaijri S, Chang C S. 2002. The EMBO Journal 21:4037-4048.
20. Marrocco K, Bergdoll M, Achard P, Criqui M C Genschik P. 2010. Current Opinion in Plant Biology 13: 631-639.
21. Mudgil Y, Shiu S H, Stone S L, Salt J N, Goring D R. 2004. Plant Physiology 134:59-66.
22. Nanjo T, Kobayashi M, Yoshiba Y, Kakubari Y, Yamaguchi-Shinozaki K, Shinozaki K. 1999. FEBS Letters 461:205-210.
23. Peng M, Hannam C, Gu H, Bi Y M, Rothstein S J. 2007. Plant Journal 50:320-337.
24. Qin F, Sakuma Y, Tran L S, Maruyama K, Kidokoro S, Fujita Y, Fujita M, Umezawa T, Sawano Y, Miyazono K., Tanokura M, Shinozaki K, Yamaguchi-Shinozaki K. 2008. Plant Cell 20:1693-1707.
25. Ryu M Y, Cho S K, Kim, W T. 2010. Plant Physiology 154:1983-1997.
26. Santner A, Estelle M. 2009. Nature 459:1071-1078.
27. Savouré A, Hua X J, Bertauche N, Montagu M V, Verbruggen N. 1997. Molecular and General Genetics 254:104-109.
28. Smalle J, Vierstra R D 2004. Annual Review of Plant Physiology 55:555-590.
29. Smirnova O G, Stepanenkoa I L, Shumnyia V K. 2011. Biology Bulletin Reviews 1:314-324.
30. Stone S L, Hauksdottir H, Troy A, Herschleb J, Kraft E, Callis J. 2005. Plant Physiology 137:13-30.
31. Stone S L, Williams L A, Farmer L M, Vierstra R D, Callis J. 2006. Plant Cell 18:3415-3428.
32. Strizhov N, Abraham E, Ökrész L, Blickling S, Zilberstein A, Schell J, Koncz C, Szabados L. 1997. Differential expression of two P5CS genes controlling proline accumulation during salt-stress requires ABA and is regulated by ABA1, ABI1 and AXR2 in Arabidopsis. 12:557-569.
33. Szabados L, Savoure A. 2010. Trends in Plant Science 15:89-97.
34. Tran L S P, Nakashima K, Sakuma Y, Osakabe Y, Qin F, Simpson S D, Maruyama K, Fujita Y, Shinozaki K, Yamaguchi-Shinozaki K. 2006. Plant Journal 49:46-63.
35. Tyers M, Jorgensen P. 2000. Current Opinion in Genetics & Development 10:54-64.
36. Vanlerberghe G C, Cvetkovska M, Wang J. 2009. Physiologia Plantarum 137:392-406.
37. Vierstra R D. 2003. Trends in Plant Science 8:135-142.
38. Vierstra R D. 2009. Nature Reviews Molecular Cell Biology 10:385-397.
39. Yan J, Wang J, Li Q, Hwang J R, Patterson C, Zhang H. 2003. A Plant Physiology 132:861-869.

Having described a preferred embodiment of the present invention, it is to be understood that variants and modifications thereof falling within the spirit of the invention may become apparent to those skilled in this art, and the scope of this invention is to be determined by appended claims and their equivalents.

What is claimed is:

1. A method for conferring drought stress tolerance on a plant, comprising:
(a) inactivating an E3 ubiquitin ligase atrzf1 in a plant cell; and
(b) obtaining the transgenic plant with enhanced drought stress tolerance from the plant cell.

2. The method according to claim 1, wherein the step (a) is carried out using T-DNA insertion into the E3 ubiquitin ligase atrzf1 consisting of the nucleotide sequence of SEQ ID NO:3.

3. A method for preparing a transgenic plant with enhanced drought stress tolerance, comprising:
(a) inactivating an E3 ubiquitin ligase atrzf1 in a plant cell; and
(b) obtaining the transgenic plant with enhanced drought stress tolerance from the plant cell.

4. The method according to claim 3, wherein the step (a) is carried out using T-DNA insertion into the E3 ubiquitin ligase atrzf1 consisting of the nucleotide sequence of SEQ ID NO:3.

5. A plant cell having drought stress tolerance, transformed with an inactivated E3 ubiquitin ligase atrzf1.

6. The plant according to claim 5, wherein the plant is selected from the group consisting of food crops, vegetable crops, crops for special use, fruit trees and fodder crops.


SEQUENCE LISTING (3 sequences)

SEQ ID NO: 1 - DNA, 1527 bp, Artificial Sequence
  AtRZF1 cDNA
aaaagactcaatagagtgtaacggagagattcttctaccagagttgctctcttttggttcgttttcttcctccaaaatccaatcttaccacagtttctttagggtttcttcttcgtcttcctttggatctatgtgaacagtatgaactaagctctgaggtaagcttgggaagtagggatatggccaaaatctctttggtttaccaacttagcaaaaaagttagcaggagttggtgatcagttctcctaccggcttttgcaggctaataactaatacctgactgtaaaatcttccaaggtttcaaccacacagggaccttgtttctcttgttattgtaaaggatgtcaagtattcggaatactcattggtgtcacagatgtcaacgtgctgtttggcttcgagctcgagatgctgtctgttcatattgtggaggagggtttgttgaggaaatcgatataggaccgagtagagctcacagggatgttgagcgtgatccaacctttgatctcatggaagctttctcagcctttatgagaagccgtttagctgaaagaagctatgaccgagaaatcagcggaagacttggctctgcgggttctgaaagcttttcaaacttggctcctctattgatctttggtggccaagctccctttcgattggctggtggtgataatagttcagttgaagcctttgtcaatggcgcagcacctggaattggtatcgcacgtggcaccaatgccggagactatttttttggacccggtcttgaagaactgattgaacagctttcctcaggcactcatcaccgaggcccaccaccagcaccgaaatcatcaattgatgcattgccaaccatcaagatcacacagaagcatctcaagtcatcagactctcactgcccggtttgcaaagacgagttcgaactgaaatcagaagcaaaacagatgccgtgtcaccatatctatcattctgactgcattgtcccgtggctggttcagcataactcatgcccagtctgtcgtaaagagttgccatccagaggatcttcttcaagcacacagagtagtcagaacagaagcaccaatggaagagaaaacagcagaagaaggaacattttctctaacctctggccattccgctcgtctagctcaagctcgactcaaaaccgcagagacacaaacaacacagcaactgcagaagaaggccactatcatcatcaccagcagcaacagcaacaacatcaacatcaacatcaacaacaacaatcccatatgggttacagtggatggccttttgactattaaaggttaatctaactcttgactctttttaagccttctgtttcactttggttctgtgtattttgtttgtgttttgtcatgtctctctttcccttactttgttcattgtttattgtagatcttctgtttctctggtttatcaactttatgtttttattttgaattgtgtatctcacacatgttagcttgttattgaatttgcattgaataaattattgttttagac

SEQ ID NO: 2 - protein, 320 aa, Artificial Sequence
  AtRZF1 amino acid sequence
MSSIRNTHWCHRCQRAVWLRARDAVCSYCGGGFVEEIDIGPSRAHRDVERDPTFDLMEAFSAFMRSRLAERSYDREISGRLGSAGSESFSNLAPLLIFGGQAPFRLAGGDNSSVEAFVNGAAPGIGIARGTNAGDYFFGPGLEELIEQLSSGTHHRGPPPAPKSSIDALPTIKITQKHLKSSDSHCPVCKDEFELKSEAKQMPCHHIYHSDCIVPWLVQHNSCPVCRKELPSRGSSSSTQSSQNRSTNGRENSRRRNIFSNLWPFRSSSSSSTQNRRDTNNTATAEEGHYHHHQQQQQQHQHQHQQQQSHMGYSGWPFDY

SEQ ID NO: 3 - DNA, 2198 bp, Artificial Sequence
  AtRZF1 genomic DNA
aaaagactcaatagagtgtaacggagagattcttctaccagagttgctctcttttggttcgttttcttcctccaaaatccaatcttaccacagtttctttagggtttcttcttcgtcttcctttggatctatgtgaacaggtatgtaactgattgagctgctctttctgattaaccaagtaaattgttctcttgaatcttatcattttattttgtttcatttttcttgttggatttagatccttggatcaaatttgctcaaatgttgtggtaaagtctgtgcctttgattgtatttcggatattttctgtgtgttttactctgatattgattaggatttgtgtatttcgtattttttttttccattgaagcgttttctgtgatctggtttttgatcgatcttattcctcttgattaaccgaacgaaagatgttttgatttctgtagatctcttgcattgtactttcttcattgttcttaattcgattttctctccattgctggatcgcctgatggctcatttagagtttttttttctagctcaaatgtggttgaactatagagtttctgagttcattatttggtcattttttaattgagttggcttgattaaagaagaatttatttgttaatcagctagattagatttgaagattgtagtagattagtatgatgtgaatgttctaatctgggaataaagggtctagtgttgagggaaacatgagatttttcataattgggtgaaatatgtgttgtaaagtttggtcttttatttgcttaattatgacttgtggttccattgactcttgtagtatgaactaagctctgaggtaagcttgggaagtagggatatggccaaaatctctttggtttaccaacttagcaaaaaagttagcaggagttggtgatcagttctcctaccggcttttgcaggctaataactaatacctgactgtaaaatcttccaaggtttcaaccacacagggaccttgtttctcttgttattgtaaaggatgtcaagtattcggaatactcattggtgtcacagatgtcaacgtgctgtttggcttcgagctcgagatgctgtctgttcatattgtggaggagggtttgttgaggaaatcgatataggaccgagtagagctcacagggatgttgagcgtgatccaacctttgatctcatggaagctttctcagcctttatgagaagccgtttagctgaaagaagctatgaccgagaaatcagcggaagacttggctctgcgggttctgaaagcttttcaaacttggctcctctattgatctttggtggccaagctccctttcgattggctggtggtgataatagttcagttgaagcctttgtcaatggcgcagcacctggaattggtatcgcacgtggcaccaatgccggagactatttttttggacccggtcttgaagaactgattgaacagctttcctcaggcactcatcaccgaggcccaccaccagcaccgaaatcatcaattgatgcattgccaaccatcaagatcacacagaagcatctcaagtcatcagactctcactgcccggtttgcaaagacgagttcgaactgaaatcagaagcaaaacagatgccgtgtcaccatatctatcattctgactgcattgtcccgtggctggttcagcataactcatgcccagtctgtcgtaaagagttgccatccagaggatcttcttcaagcacacagagtagtcagaacagaagcaccaatggaagagaaaacagcagaagaaggaacattttctctaacctctggccattccgctcgtctagctcaagctcgactcaaaaccgcagagacacaaacaacacagcaactgcagaagaaggccactatcatcatcaccagcagcaacagcaacaacatcaacatcaacatcaacaacaacaatcccatatgggttacagtggatggccttttgactattaaaggttaatctaactcttgactctttttaagccttctgtttcactttggttctgtgtattttgtttgtgttttgtcatgtctctctttcccttactttgttcattgtttattgtagatcttctgtttctctggtttatcaactttatgtttttattttgaattgtgtatctcacacatgttagcttgttattgaatttgcattgaataaattattgttttagac